(19) United States
(12) Patent Application Publication
(10) Pub. No.: US 2026/0309695 A1
(43) Pub. Date: Oct. 8, 2026

(54) Method and System for Continuous Analyte Concentration Monitoring with Integrated Patient Specific Deficiency Classification and Recording Processor

(71) Applicant: Omaroon Corporation, Orange, CA (US)

(72) Inventor: Louay Khaled Fakhro, Mission Viejo, CA (US)

(21) Appl. No.: 19/638,753

(22) Filed: Apr. 3, 2026

Related U.S. Application Data
(60) Provisional application No. 63/782,890, filed on Apr. 3, 2025.

(51) Int. Cl. G01N 33/487; G01N 33/68

(52) U.S. Cl. CPC G01N 33/48785; G01N 33/6812

(57) ABSTRACT

Systems, devices, and methods directed to continuous analyte concentration monitoring with integrated patient-specific deficiency classification are disclosed. An integrated circuit chip includes an analog front end cell having an amplifier cell in communication with a continuous analyte concentration electrode of a non-invasive sensor and an analog signal processing unit cell, a digital back end channel controller cell, a feature extraction engine processor cell, and a classification engine cell comprising a patient-specific support vector machine. A signal received from the continuous analyte concentration electrode is amplified, adapted, digitized, and processed to divide the signal into a plurality of sub-bands, determine respective sub-band energies, and aggregate the respective sub-band energies into a feature vector. The classification engine cell designates the feature vector as representing an amino acid deficient condition or an amino acid non-deficient condition.

### CROSS-REFERENCE TO RELATED APPLICATIONS

This application claims the benefit of priority to U.S. Provisional Patent Application No. 63/782,890, filed Apr. 3, 2025, the entire contents of which is incorporated by reference herein.

### BACKGROUND

Tracking amino acid deficiency in order to provide proper diagnosis is an important process for patients to manage such a deficiency on a moment to moment basis. Non-invasive measuring of disease parameters by spectrophotometric sensors has been attempted, but these approaches have encountered the problem of a medical practitioner locating an area of interest and being able to accurately and precisely position a sensor over the area of interest.

Accordingly, there is a need in the art for a non-invasive, real time method and system that monitors amino acid levels of a human body and that is provided with sensors which can be precisely positioned over an area of interest in order to assist in assessing conditions associated with a disease.

### SUMMARY

The present disclosure provides a system for monitoring analyte concentration levels. In one aspect, the system comprises an integrated circuit chip configured to classify data representing a level of an amino acid as representing an amino acid deficient condition or an amino acid non-deficient condition. In several embodiments, the integrated circuit chip comprises an analog front end cell including an amplifier cell in communication with a continuous analyte concentration electrode of a non-invasive sensor and an analog signal processing unit cell in communication with the amplifier cell; a digital back end channel controller cell; a feature extraction engine processor cell in communication with the digital back end channel controller cell and the analog front end cell; and a classification engine cell in communication with the feature extraction engine processor cell and the digital back end channel controller cell. In several embodiments, the classification engine cell comprises a patient-specific support vector machine configured to process a signal received from the continuous analyte concentration electrode and designate the signal as representing an amino acid deficient condition or an amino acid non-deficient condition. In certain embodiments, the system further comprises an output component configured to output a recommendation to administer amino acid if the integrated circuit chip classifies the measurement data as representing the amino acid deficient condition, or a recommendation not to administer amino acid if the integrated circuit chip classifies the measurement data as representing the amino acid non-deficient condition.

In another aspect, the present disclosure provides a method for monitoring a continuous analyte concentration using an integrated circuit chip is contemplated. In several embodiments, the method comprises amplifying a signal from a continuous analyte concentration electrode of a non-invasive sensor to generate an amplified analyte concentration signal; adapting gain of the amplified analyte concentration signal using an analog signal processing unit to generate an adapted gain signal; digitizing the adapted gain signal using an analog to digital converter; extracting a feature vector from the digitized adapted gain signal; designating, with a patient-specific support vector machine classification engine of the integrated circuit chip, the feature vector as representing an amino acid deficient condition or an amino acid non-deficient condition; and outputting a recommendation to administer amino acid if the integrated circuit chip classifies measurement data as representing the amino acid deficient condition, or a recommendation not to administer amino acid if the integrated circuit chip classifies the measurement data as representing the amino acid non-deficient condition.

In another aspect, the present disclosure provides a device. In several embodiments, the device comprises an integrated circuit chip for continuous analyte concentration monitoring. The integrated circuit chip comprises an analog front end cell including an amplifier cell in communication with a continuous analyte concentration electrode of a non-invasive sensor and an analog signal processing unit cell in communication with the amplifier cell; a digital back end channel controller cell; a feature extraction engine processor cell in communication with the digital back end channel controller cell and the analog front end cell; and a classification engine cell in communication with the feature extraction engine processor cell and the digital back end channel controller cell. In several embodiments, the classification engine cell comprises a patient-specific support vector machine configured to process a signal received from the continuous analyte concentration electrode and designate the signal as representing an amino acid deficient condition or an amino acid non-deficient condition. In certain embodiments, the device further comprises an output component configured to output a recommendation to administer amino acid if the integrated circuit chip classifies the measurement data as representing the amino acid deficient condition, or a recommendation not to administer amino acid if the integrated circuit chip classifies the measurement data as representing the amino acid non-deficient condition.

Tracking amino acid deficiency in order to provide proper diagnosis requires a device having a small form factor, and ultra-low power sensors with continuous analyte concentration classification. Technical challenges arise from 1) patient-to-patient variation of an amino acid deficiency pattern in analyte concentrations, 2) fully integrating ultra-low power variable dynamic range instrumentation circuits with an amino acid deficiency detection processor, and/or 3) reducing communication overhead. Prior attempts successfully extracted analyte concentration features locally on-chip to reduce the data being transmitted, and saved power by 1/14 when compared to raw analyte concentration data transmission. However, such approaches still needed data transmission and off-chip classification to detect and to store amino acid deficiency data.

What is needed, and is provided for in embodiments herein, is an ultra-low power scalable analyte concentration acquisition System on a Chip (SoC) for continuous amino acid deficiency detection and recording with a fully integrated patient-specific Support Vector Machine (SVM) based classification processor. Unless otherwise stated to the contrary, the term “SOC” shall refer to a printed circuit board comprising a plurality of integrated chips functionally integrated therewith.

Non-invasive measuring of disease using near infrared sensors, such as spectrophotometric sensors, to measure amino acid levels within a human body has been attempted. However, such conventional techniques have encountered the problem of a medical practitioner locating an area of interest and being able to accurately and precisely position a sensor over the area of interest. Often the orientation of the scan and the depth of the scan produced by a near infrared sensor as well as the orientation of the area of interest can be challenging for a medical practitioner to determine because conventional sensors are not marked with any instructions or visual aids.

Accordingly, there is a need in the art for a non-invasive, real time method and system that monitors amino acid levels of a human body and that is provided with sensors which can be precisely positioned over an area of interest in order to assist in assessing conditions associated with a disease. A further need exists in the art for a non-invasive method that monitors amino acid levels of an area of interest over long periods of time at frequent time intervals and that can monitor different areas that may be in close proximity with one another. Another need exists in the art for amino acid detecting sensors that can be fabricated to fit the size of the area of interest, often wearable by a patient. There is also a need in the art for a non-invasive method and system that monitors amino acid levels and that can identify ideal locations along a human body in which to collect various samples for comparison. There is another need in the art for sterile, non-invasive amino acid detecting sensors that can be used under surgical and operating conditions. There is a need for multiple locations and multiple areas of interest to be monitored in a continual and orchestrated manner by a single system. In other words, multiple monitors coordinated to limit noise and continually monitor multiple areas of interest are needed in the art.

The present disclosure provides embodiments that address the above needs.

Several embodiments of the invention provided for herein relate to an integrated circuit chip for continuous analyte concentration (CAC) monitoring. In one embodiment, the integrated circuit chip for CAC monitoring includes: an Analog Front End cell, the Analog Front End cell includes: an amplifier cell in communication with a continuous analyte concentration electrode (CAC electrode) of a non-invasive sensor; an analog signal processing unit (ASPU) cell in communication with the amplifier cell; and a Classification Processor including: a digital back end (DBE) Channel Controller cell; a Feature Extraction Engine Processor cell, the Feature Extraction Engine Processor cell in communication with the DBE Channel Controller cell and the Analog Front End cell; and a Classification Engine cell in communication with the Feature Extraction Engine Processor cell and the DBE Channel Controller cell, wherein a signal received from the CAC electrode is processed by the Classification Engine cell and designated as amino acid deficient or amino acid non-deficient. In another embodiment, the amplifier cell in communication with the electrode is a Chopper Stabilized-Capacitive Coupled Instrumentation Amplifier (CS-CCIA) cell. In yet another embodiment, the amplifier cell in communication with the CAC electrode is an autozeroing amplifier circuit cell. In still yet another embodiment, the Feature Extraction Engine Processor cell includes: a plurality of bandpass filter cells, each bandpass filter cell in communication with the ASPU cell; and a plurality of integrator cells, each integrator cell in communication with a respective one of the plurality of bandpass filter cells; wherein a signal from the ASPU cell of an Analog Front End is filtered by a plurality of bandpass filters to form a plurality of sub-bands, and wherein each of the sub-bands is integrated by a respective one of the plurality of integrator cells to determine the energy of the sub-band.

In one embodiment, the Feature Extraction Engine Processor cell further includes: a decimation filter cell in communication between the ASPU cell and the plurality of bandpass filter cells, and wherein a signal from the ASPU cell of an Analog Front End is decimated by the decimation cell prior to being filtered by the plurality of bandpass filter cells. In another embodiment, the integrated sub-bands are aggregated to form a feature vector. In yet another embodiment, Classification Engine cell designates the signal received from the CAC electrode as an amino acid deficiency condition or an amino acid non-deficiency condition in response to the feature vector. In still yet another embodiment, the integrated circuit chip further includes an analog to digital converter cell in communication with the ASPU cell and the Feature Extraction cell. In one embodiment, the analog to digital converter cell in communication with the ASPU cell and the Feature extraction cell is a Successive-approximation-register (SAR) analog-to-digital converters (ADC). In another embodiment, the Classification Engine includes an SVM and the SVM designates the signal, received from the electrode, as amino acid deficient or amino acid non-deficient in response to the feature vector, by determining on which side of a hyperplane in feature hyperspace the feature vector lies. In yet another embodiment, the Analog Front End cell is a plurality of Analog Front End cells, wherein the Feature Extraction Engine cell is a plurality of Feature Extraction Engine cells, and wherein each Feature Extraction Engine cell is in communication with a respective one of the Analog Front End cells. In still yet another embodiment, the integrated circuit chip further includes: a multiplexer cell in communication with the plurality of Analog Front End cells; and an ADC cell in communication with the multiplexer cell and the plurality of Feature Extraction Engine processor cells. In another embodiment, the ADC cell is an SAR cell.

In yet another embodiment, the DBE Channel controller includes: a channel gain control line and a channel bandwidth control line in communication with an Analog Front End cell. In still yet another embodiment, the DBE Channel Controller includes a channel gain control line, a channel bandwidth control line, and a channel number select in communication with the Analog Front End (AFE)_Channel Controllers and a channel select control line in communication with the multiplexer cell, wherein the DBE Channel Controller is in communication with an external data interface, and wherein the DBE Channel Controller selects the number of channel to be used in response to receiving the number of channels from the external data interface and setting the respective AFE Channel Controller on or off. In another embodiment, each bandpass filter comprises: a plurality of Look Up Tables (LUTs), each LUT having a plurality of inputs and having an output, a plurality of bit shift registers, each bit shift register having an input in communication with the ASPU and an output in communication with a respective input of the LUT; a summer having a first input in communication with the output of the LUT and having a second input and an output; and a register having an input in communication with the output of the summer and having an output in communication with the second input of the summer; wherein the output of the bandpass filter is the output of the register. In yet another embodiment, LUT is a distributed quad-LUT. In still yet another embodiment, the CS-CCIA includes: an input-chopped switch having an input in communication with the electrode and having an output; a low noise amplifier having an input in communication with the output of the input-chopped switch and an first output connected to an input of the ASPU and having a second output; an Impedance Boosting Loop including a IBL chopper switch having an input in communication with the low noise amplifier output and an output in communication with the low noise amplifier input; a Direct Current (DC) Servo Loop including: a DC Servo Loop (DSL) amplifier having an input in communication with the output of the low noise amplifier; a DSL chopper switch having an input in communication with the output of the DSL amplifier and a output in communication with the input of the low noise amplifier; and an Offset Cancellation Loop (OCL) including: an OCL amplifier having an input in communication with the second output of the low noise amplifier and having an output; an OCL chopped switch having an input in communication with the output of the OCL amplifier and having an output; a high pass filter having an input in communication with the output of the OCL chopped switch and having an output; and an gain chopped switch having an input in communication with the output of the high pass filter and having an output in communication with the input of the low noise amplifier.

In one embodiment, the autozeroing amplifier circuit cell includes: an auto zero amplifier having an input in communication with the electrode and having an output; a low noise amplifier having an input in communication with the output of the autozero amplifier and an first output connected to an input of the ASPU and having a second output; an Impedance Boosting Loop including a IBL chopper switch having an input in communication with the low noise amplifier output and an output in communication with the low noise amplifier input; a DC Servo Loop including: a DSL amplifier having an input in communication with the output of the low noise amplifier; a DSL chopper switch having an input in communication with the output of the DSL amplifier and a output in communication with the input of the low noise amplifier; and an Offset Cancellation Loop including: an OCL amplifier having an input in communication with the second output of the low noise amplifier and having an output; an OCL chopped switch having an input in communication with the output of the OCL amplifier and having an output; a high pass filter having an input in communication with the output of the OCL chopped switch and having an output; and an gain chopped switch having an input in communication with the output of the high pass filter and having an output in communication with the input of the low noise amplifier. In another embodiment, the integrated circuit chip further includes: a bus; a Static random-access memory (SRAM) in communication with the bus; an external memory interface in communication with the bus; and an external data interface in communication with the bus; wherein the bus is in communication with the Classification Processor.

In another aspect, the invention relates to a method for monitoring a continuous analyte concentration using an integrated circuit chip. In one embodiment, the method for monitoring a continuous analyte concentration using an integrated circuit chip includes the steps of: amplifying a signal from an CAC electrode of a non-invasive sensor to generate an amplified analyte concentration signal; adapting the gain of the amplified analyte concentration signal using an ASPU; digitizing the adapted gain signal using an analog to digital converter; extracting a feature vector from the digitized adapted gain signal using a feature extraction engine; and designating the feature vector as amino acid deficient or amino acid non-deficient using a classification engine. In another embodiment, the step of adapting the gain of the amplified analyte concentration signal occurs in real-time. In another embodiment, the step of extracting a feature vector includes the steps of: dividing the digitized adapted gain signal into a plurality of sub-bands; integrating each sub-band to calculate the energy within each sub-band; and aggregating each integrated sub-band to generate a feature vector. In still yet another embodiment, the step of designating the feature vector as amino acid deficient or amino acid non-deficient includes the steps of: generating, in response to patient data, a hyperplane separating a deficient region of feature hyperspace from a non-deficient region of feature hyperspace; and determining in which region of feature hyperspace the feature vector exists. In another embodiment, the step of dividing the digitized adapted gain signal into a plurality of sub-bands utilizes a LUT.

A method and system for monitoring amino acid levels in a human body, can be used to assist in the diagnosis of a disease. The method and system can include one or more near infrared non-invasive sensors in which each sensor can be provided with an alignment mechanism and a central scan depth marker so that each sensor may be precisely positioned over a human leg or human thigh or forearm or wrist or finger (or other appropriate limb or region of the body). The method and system can include a device for displaying amino acid levels corresponding to respective non-invasive sensors that are measuring amino acid levels of a patient.

The alignment mechanism of a non-invasive sensor can include a linear marking on a surface of the non-invasive sensor that is opposite to the side which produces a light scan used to detect amino acid levels. The linear marking can be used by a medical practitioner to align a non-invasive sensor with the longitudinal axis of, for example, a patient limb or region of interest.

The central scan depth marker can include a linear marking positioned on a surface of a non-invasive sensor that intersects the alignment mechanism, a crosshatch, at a location along the alignment mechanism that denotes the deepest region of a light scan produced by the non-invasive sensor. The depth of measurement can be displayed in numeric form over the crosshatch guide to aid the medical practitioner since depth varies based on light source & receptor separation. The central scan depth marker can ensure that a medical practitioner properly aligns the non-invasive sensor at a location that will measure an area of interest.

According to another further non-limiting example embodiment, the inventive system incorporates amino acid levels of other areas of the body or different sensors for different functions. Initial data from a patient with a suspected disease can be measured via, for example, amino acid levels in a diseased sample. If a disease is suspected, the amino acid levels can be monitored and would be deficient from an elevated state to an equal and then lower level with comparison to a healthy sample (or vice versa). Therefore, when comparing the diseased sample and the healthy sample, the diseased sample should show decreased amino acid levels. If levels begin to drop in the diseased sample compared to the healthy sample, an alarm or alert can be triggered to warn the patient and/or medical practitioner. A display for the amino acid levels being measured can also be provided by the system.

According to an embodiment of the invention, a method of performing real-time sensor diagnostics on a subcutaneous, implanted or non-invasive sensor having at least one CAC electrode, comprises performing a first electrochemical impedance spectroscopy (EIS) procedure to generate a first set of impedance-related data for the at least one CAC electrode; after a predetermined time interval, performing a second EIS procedure to generate a second set of impedance-related data for the at least one CAC electrode; and, based solely on the first and second sets of impedance-related data, determining whether the sensor is functioning normally.

In accordance with another embodiment of the invention, a method of calculating a single, fused sensor glucose value is disclosed. The fused sensor glucose value is calculated based on glucose measurement signals from a plurality of redundant sensing CAC electrodes by performing respective electrochemical impedance spectroscopy (EIS) procedures for each of the plurality of redundant sensing electrodes to obtain values of at least one impedance-based parameter for each the sensing CAC electrode; measuring the electrode current (Isig) for each of the plurality of redundant sensing CAC electrodes; independently calibrating each of the measured Isigs to obtain respective calibrated sensor glucose values; performing a bound check and a noise check on the measured Isig and the values of the at least one impedance-based parameter and assigning a bound-check reliability index and a noise-check reliability index to each of the sensing CAC electrodes; performing signal-dip analysis based on one or more of the at least one impedance-based parameter and assigning a dip reliability index to each of the sensing CAC electrodes; performing sensitivity-loss analysis based on one or more of the at least one impedance-based parameter and assigning a sensitivity-loss index to each of the sensing CAC electrodes; for each of the plurality of electrodes, calculating a total reliability index based on the CAC electrode's bound-check reliability index, noise-check reliability index, dip reliability index, and sensitivity-loss reliability index; for each of the plurality of electrodes, calculating a weight based on the CAC electrode's total reliability index; and calculating the fused sensor glucose value based on the respective weights and calibrated sensor glucose values of each of the plurality of redundant sensing CAC electrodes.

In yet a further embodiment of the invention, a method is disclosed for real-time monitoring of sensitivity loss for a CAC electrode of a non-invasive sensor by periodically performing an electrochemical impedance spectroscopy (EIS) procedure to generate multiple sets of impedance-related data for the CAC electrode; calculating values of one or more impedance-related parameters based on the multiple sets of impedance-related data; monitoring the values over time; and, based on the values, determining whether the CAC electrode is experiencing sensitivity loss.

In accordance with yet another embodiment of the invention, a CAC sensing system includes a subcutaneous or implanted sensor having a plurality of independent CAC electrodes, a counter electrode, and a reference electrode, and sensor electronics operably coupled to the sensor. The sensor electronics, in turn, include electronic circuitry configured to selectively perform an electrochemical impedance spectroscopy (EIS) procedure for one or more of the plurality of independent CAC electrode to generate impedance-related data for the one or more CAC electrodes; a programmable sequencer configured to provide a start stimulus and a stop stimulus for performing the EIS procedure; and a microcontroller interface configured to operably couple the sensor electronics to a microcontroller.

Accordingly, in a first aspect a wholly implantable analyte sensor system or other type of implantable sensors, is provided comprising a wholly implantable body comprising an electrode configured to measure a glucose level in a host; a membrane disposed over the electrode and configured to limit transport of glucose to the electrode; a layer comprising an enzyme to catalyze a reaction of glucose and oxygen as a co-reactant; and a sensor electronics unit operably connected to the electrode and configured to measure a current produced by the electrode, wherein the sensor electronics unit is configured to measure glucose concentration with substantial linearity at glucose concentrations of up to about 400 mg/dL at an oxygen concentration of less than about 0.6 mg/L.

In a second aspect, a device for measuring a concentration of glucose in a biological fluid is provided, the device comprising a housing comprising an electronic circuit and at least one electrode operatively connected to the electronic circuit, wherein the electrode is configured to determine, in operation, a concentration of glucose in a biological sample; and a glucose determining apparatus operatively connected to the electrode and comprising a membrane impregnated with an oxidase, wherein the electronic circuit is configured to measure, in operation, a current representative of the concentration of glucose in the biological sample, wherein the glucose determining apparatus is configured to measure glucose concentration with substantial linearity at glucose concentrations of up to about 400 mg/dL at an oxygen concentration of less than about 0.6 mg/L.

### BRIEF DESCRIPTION OF THE DRAWINGS

Embodiments of the invention are illustrated by way of example, and not by way of limitation, in the figures of the accompanying drawings in which like reference numerals refer to similar elements.

FIG. 1 is a block diagram of an embodiment of the scalable continuous analyte concentration (CAC) System on a Chip (SoC) 100 constructed in accordance with a non-limiting embodiment of the invention.

### DETAILED DESCRIPTION

In the following detailed description of embodiments, reference is made to the accompanying drawings in which like references indicate similar elements, and in which is shown by way of illustration manners in which specific embodiments may be practiced. These embodiments are described in sufficient detail to enable those skilled in the art to practice the embodiments of the invention provided for herein, and it is to be understood that other embodiments may be utilized and that logical, mechanical, electrical, functional and other changes may be made without departing from the scope of the present disclosure. The following detailed description is, therefore, not to be taken in a limiting sense, and the scope of the present invention is defined only by the appended claims.

The system architectures, component arrangements, processing blocks, and communication pathways described herein are provided as illustrative examples of implementations of the disclosed subject matter and should not be construed as requiring any particular configuration, sequence, or combination of elements unless expressly recited otherwise. A person of ordinary skill in the art will appreciate that functions described with respect to particular cells, engines, controllers, modules, circuits, or other components may be combined, separated, reordered, distributed, omitted, or implemented in alternative architectures while remaining within the scope of the present disclosure.

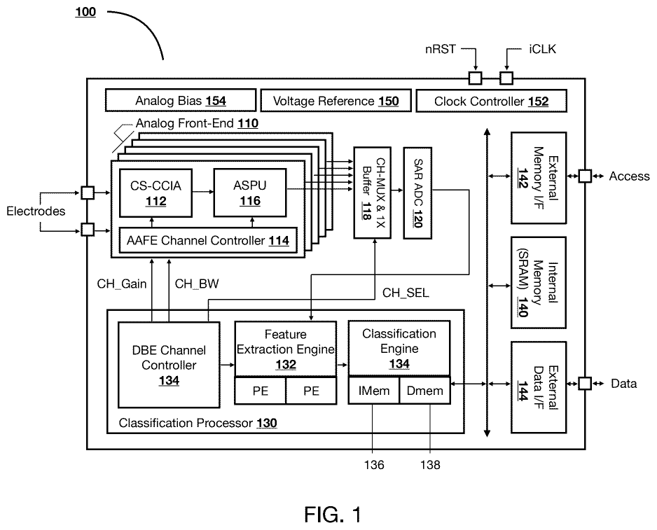

In brief overview, FIG. 1 discloses a block diagram of an embodiment of the scalable continuous analyte concentration (CAC) System on a Chip (SoC) 100 constructed as interconnected integrated circuit cells (generally “cells”) in accordance with the invention. This non-limiting embodiment includes eight Analog Front-End processor cells (collectively 110). Two electrodes, configured as a differential pair, connect to one Analog-Front-End to form one channel. In several embodiments, the eight Analog Front-End cells accommodate a total of 16 electrodes. However, this is not meant to be limiting or required as a fixed value, as in some embodiments, the Analog Front-End includes one or more cells and the one or more cells accommodate a total of any number of electrodes, such as 2-22 electrodes (or any number therebetween). A larger number of electrodes is used in additional embodiments (e.g., where a larger area is used). Each Analog Front-End 110 cell includes a continuous-time Chopper Stabilized-Capacitive Coupled Instrumentation Amplifier (CS-CCIA) 112, an AFE Channel Controller 114, and a digitally-assisted reconfigurable Analog Signal Processing Unit (ASPU) 116. Each Analog Front-End cell 110 is connected to a respective input of a multiplexer cell 118. The output terminal of the multiplexer 118 is connected to the input of a ten bit SAR ADC cell 120 digitizing at a rate of 4K samples/sec/channel and whose output value is an input value to a patient-specific machine-learning amino acid deficient Classification Processor 130. The Classification Processor 130 includes a number of Feature Extraction Engine (FEE) processor cells 132, a Digital Back End (DBE) Channel Controller cell 134 and a Classification Engine (CE) cell 134. The CE 134 includes internal system memory interfaces (IMem) 136 for system instructions and (DMem) 138 for cache or data access to SRAM 140 and external memory, as described below. The IMem 136 and DMem 138 allow the SoC 100 to see both internal memory 140 and optional external memory as if they were a single unified storage.

In several embodiments, the SoC 100 includes an SRAM cell 140 for storing data, an external memory interface cell 142 for allowing access to external memory and an external data interface cell 144 to permit external access to the data memory 140, debugging and the setting of certain system parameters, such as the number of channels, using configuration pins in the external data interface 144. These three cells are in communication with a bus that is in turn in communication with the Classification Processor 130 generally, and the DBE Controller 134, Feature Extraction Engine 132 and Classification Engine 134, specifically. The SoC 100 further has a voltage reference cell 150 that provides reference voltages to the SAR ADC 120, a clock controller cell 152 that divides the external one MHz clock signal to provide the necessary clock signals to the other cells and an analog bias circuit cell 154 to provide accurate bias voltages to the Analog Front-Ends 110.

In several embodiments, three control lines, Channel Gain (CH_GAIN), Channel Bandwidth (CH_BW) and Channel Select (CH_SEL), provide input values to both the Analog Front-End (AFE) controller 114 and channel selection in the multiplexer 118 respectively. These control lines are set by DBE Channel Controller 134 of the Classification Processor 130. In addition, the number of channels actually to be used (1, 2, 4, or 8, or another value) is set using the configuration pins of the external data interface 144 that in turn set values for the number of channels in the DBE Channel Controller 134. The DBE Channel Controller 134 then sets the AFE Channel Controller for the unused channels to “OFF” through a channel number select line. In several embodiments, the system is configured to use the number of channels as needed in ascending order. That is if 4 channels are required the first 4 channels (1-4) are used. Other embodiments use different channel configurations.

In operation, generally, each analyte concentration signal from a respective differential pair of a group of electrodes attached to a limb or region of interest of a patient is an input signal to a respective one of Analog Front-End processor cells 110 through the CS-CCIA cell 112. The CS-CIA is a low noise amplifier with a 4 KHz bandwidth described in more detail below. The resulting amplified analyte concentration signal is an input signal to the ASPU 116. The ASPU 116 adapts the gain of the signal and limits the bandwidth of the analyte concentration signal to 4 KHz. The output signals of all eight of the ASPU cells 110, each of which is also referred to as a channel, pass to the multiplexer 118 which selects the data channel to be passed to the SAR ADC 120.

One of the channels at a time is selected by a channel select CH_SEL control line asserted by the DBE Channel Controller 134. The selected channel data is digitized by the SAR ADC cell 120 using a portion of the time allowed for sampling all eight Analog Front-End 110 cells at 300 Hz-4 kHz/channel. For example, the selected channel data is digitized by the SAR ADC cell 120 using ⅛ of the time allowed for sampling all eight Analog Front-End 110 cells at 4 KHz/channel. The SAR samples at 4 KHz/sec/channel and will increase its digitization rate to meet this requirement. The multiplexer 118 will increase its scan rate of the channels to track the rate of digitization by the SAR 120. The 10 bit digitized value of the analyte concentration signal from the SAR ADC 120 is the input value to the Feature Extraction Engine (FEE) (generally 132) through a classification multiplexer (not shown). In some embodiments, the digitized value of the analyte concentration signal is more or less than 10 bits, such as 1-24 bits. The classification multiplexer takes the value of the sampled channel from the SAR ADC 120 and passes it to one of the processor elements (only two shown for clarity in FIG. 1) which make up the FEE 132. That is, each channel is assigned to and provides input data to one FEE processor element.

In one embodiment, the CS-CCIA is replaced with an autozeroing amplifier circuit cell (not shown) which is identical to the CS-CCIA except that rather than modulate the analyte concentration signals using the chopper MOD in 112, the chopper MOD in 112 is replaced with an autozero amplifier between the electrodes and the Cin capacitors (not shown in FIG. 1).

By way of description only, as other embodiments employ different data amounts, the 10 bits of data from the selected channel entering the Feature Extraction Engine processor element 132 from the SAR ADC 120 are decimated by a decimation filter. In one embodiment, the decimation filter down-samples the 4K samples/sec being provided by the SAR ADC 120 by eight to produce 500 samples/sec of signal that is stored in the SRAM 140 and by 142 to produce the 128 samples/sec that will be utilized by the FEE processor element 132. In other embodiments, the decimation filter down-samples the 4K samples/sec by more of less than eight (other down or up-sampling rates are used in other embodiments). This is done because the data rate of the SAR ADC 120 over samples compared to what is necessary for feature extraction. In other embodiments, the SAR ADC 120 is replaced with a standard ADC. Further, one embodiment does not use the decimation filter and instead connects the bandpass filters directly to the SAR ADC 120 or the ADC.

The data from the decimation filter is passed through seven bandpass filters (BPF) each of which passes a different 4 Hz of sample, termed a sub-band. That is, one BPF passes 0-4 Hz sub-band, the second passes 4-8 Hz sub-band and so on. Each sub-band is then passed to a respective one of a series of integrators which integrates the sub-band over a 2 second interval to obtain the energy of the sub-band. The 10 bits of data from each sub-band are aggregated with the other sub-band data into 70 bits of channel data that is a feature vector for that channel. This feature vector is a feature vector without temporal information. By applying a triplet sliding window of the feature vector over time, an extracted feature vector (X) is formed that does have temporal information. The channel feature vectors is channel data and the channel data from the other seven FEE processors 130 are input to a multiplexer 118 of the CE 134. Depending on the embodiments, other bandpass filter configurations can be used.

The feature vector from each channel is input to the Support Vector Machine 134. The SVM also receives input from the classifier which has been trained using patient specific data 120. The SVM designates incoming feature vector from a channel as a amino acid deficient or amino acid non-deficient state by determining where the data falls relative to a six dimensional (corresponding to the seven frequency sub-bands) hyperplane that separates deficient or non-deficient regions of feature hyperspace. The SVM 134 classifies the incoming vector based in part on the patient specific parameters determined during training using data stored off-chip in a patient specific database. When an amino acid deficient condition is detected, the SVM sets a control line, CH_SEZ, high. This control line can then be used as a trigger for an alarm or other notification purpose.

It should be noted that, according to some embodiments, one electrode, or other numbers, rather than a differential pair may be used. Further if less data is required, the SOC 100 can be constructed to measure an analyte concentration with only one electrode rather than the sixteen electrodes just discussed thereby reducing the number of channels to one (or another amount configured based on the alternative number of electrodes). In such a case, there is only one Analog Front End 110 required. Therefore, in this non-limiting example, there is no multiplexer 118 required and the SAR DAC 120 can be connected to the output of the ASPU 116. Because there is only one channel, the Feature Extraction Engine 132 requires only one Feature Extraction Engine Processor.

In some embodiments, the Analog Front-End 110 cell may be used as an Electro-chemical sensor and may follow a standard 3 electrode electro-chemistry setup. In some embodiments, the 3 electrode setup could be made up of a plurality of microneedles. In some embodiments, the microneedles could be a setup of between seven (7) and eleven (11) microneedles. In some embodiments, a plurality of microneedles could have different groupings that perform an electro-chemical approach without blood being drawn or based on a test of sweat.

In some embodiments, the SOC 100 may further include a test strip, or a plurality of test strips, used to measure amino acids depending on the chemistry contents. In a preferred embodiment, the test strip can measure amino acids based on colorimetric methods where the color changes based on chemical interactions. In a preferred embodiment, the test strip can be designed to use colorimetric techniques in higher concentration ranges. In some embodiments, the test strip may be used to measure all twenty (20) amino acids, including but not limited to: Tyrosine and its downstream metabolites, branched-chain amino acids (BCAAs) which may be leucine, isoleucine, and valine. In some embodiments, the test strip may be used to measure of all twenty (2) amino acids for the monitoring of, including but not limited to: glucose, ketones, ammonia, lactate, homocysteine, cysteine, methionine, arginine and citrulline, glutathione, organic acids. In some embodiments, the test strip may be used to measure all twenty (20) amino acids for the monitoring of diseases, including but not limited to: phenylketonuria (PKU), maple syrup urine disease (MSUD), tyrosinemia, and homocystinuria.

In some embodiments, the SOC 100 may further include a Near Field Communication (NFC) cell (not shown in FIG. 1) in communication with the test strip. In some embodiments, the NFC cell can serve as a power means for the test strip. In some embodiments, the NFC cell can interrogate the contents of a unique identification mark for preventing counterfeit situations. In a preferred embodiment, the NFC cell can communicate over Near Field Communication the blood levels of any amino acid, depending on the chemistry contents of the test strip, to the Classification Processor.

In preferable embodiments, a method and system for monitoring amino acid levels to assist in the diagnosis of a disease. The method and system can include one or more near infrared non-invasive sensors in which each sensor can be provided with an alignment mechanism and a central scan depth marker so that each sensor may be precisely positioned over a human leg or human thigh or forearm or wrist or finger. The method and system can include a computing device for monitoring amino acid levels to assist in the diagnosis of a disease and displaying amino acid levels corresponding to respective non-invasive sensors that are measuring amino acid levels of a patient.

In some embodiments, amino acid levels of a human body can be measured by a near-infrared spectroscopy (NIRS) sensor that include an alignment mechanism and central scan depth markers according to one non-limiting embodiment of the invention.

The alignment mechanism of a non-invasive sensor can include a linear marking on a surface of the non-invasive sensor that is opposite to the side which may produce a light scan used to detect amino acid levels. The linear marking can be used by a medical practitioner to align a non-invasive sensor with the longitudinal axis of, for example, a patient limb of interest. The invention is not limited to a solid line on the sensor. Other alignment mechanisms within the scope of the invention include, but are not limited to, tick marks, dashed lines, notches cut in the substrate of the non-invasive sensor to provide a geometric reference for the medical practitioner, and other like visual orienting alignment mechanisms.

The central scan depth marker can include a linear marking positioned on a surface of a non-invasive sensor that intersects the alignment mechanism at a location along the alignment mechanism that denotes the deepest region of a light scan produced by the non-invasive sensor. The depth of measurement can be displayed in numeric form over the central scan depth marker as a guide to aid a medical practitioner since scan depth can vary based on the non-invasive sensor's light source and receptor separation. The central scan depth marker can ensure that a medical practitioner properly aligns the non-invasive sensor at a location that will measure an area of interest. Similar to the alignment mechanism noted above, the invention is not limited to a solid line on the non-invasive sensor. Other central scan depth markers within the scope of the invention include, but are not limited to, tick marks, dashed lines, notches cut in the substrate of the non-invasive sensor to provide a geometric reference for the medical practitioner, and other like visual orienting central depth markers.

Once the proper position for a non-invasive sensor is determined by the medical practitioner with the compartment alignment mechanism and the central scan depth marker, the medical practitioner can apply the non-invasive sensor on the patient by using an adhesive that is already part of the non-invasive sensor.

In a preferred embodiment, three non-invasive sensors of a system for monitoring three different parts of a, for example, lower human leg. A fourth non-invasive sensor can be positioned on a side of the leg which monitors the fourth part of the lower human leg (or other region or limb of interest). The non-invasive sensors discussed throughout this document can be of the type described in U.S. Pat. No. 6,615,065 issued in the name of Barrett et al. (the “'065 patent”), the entire contents of which are hereby incorporated by reference. The non-invasive sensors can include those made and distributed by Somanetics, Troy, Mich. However, other conventional near infrared non-invasive sensors can be used without departing from the scope and spirit of the invention.

The non-invasive sensors can generally provide electrochemical in vivo monitoring of amino acids such as analyte concentration on a continuing and substantially instantaneous basis.

The non-invasive sensors are coupled to a processor and display unit which displays the amino acid levels. The processor and display unit can display all amino acid levels of the human body when at least three non-invasive sensors are deployed. The invention is not limited to three non-invasive sensor embodiments. Embodiments of the invention can include any number of non-invasive sensors and an Electronics cell (not shown in FIG. 1) for the accurate detection of conditions that may be associated with disease. For example, another non-limiting embodiment, allows for eight sensor readings so that monitoring of a healthy sample can be performed. In some embodiments, the invention can include any number of non-invasive sensors for the accurate detection of all twenty (20) amino acids, including but not limited to: Tyrosine and its downstream metabolites, branched-chain amino acids (BCAAs) which may be leucine, isoleucine, and valine. In some embodiments, the invention can include any number of non-invasive sensors for the accurate detection of all twenty (2) amino acids for the monitoring of, including but not limited to: glucose, ketones, ammonia, lactate, homocysteine, cysteine, methionine, arginine and citrulline, glutathione, organic acids. In some embodiments, the invention can include any number of non-invasive sensors for the accurate detection of all twenty (20) amino acids for the monitoring of diseases, including but not limited to: phenylketonuria, maple syrup urine disease, tyrosinemia, and homocystinuria.

In a non-limiting embodiment, the non-invasive sensors are coupled to the processor and display unit with cables which can include electrical conductors for providing operating power to the light sources of the non-invasive sensors and for carrying output signals from the detectors of the sensors to the display unit. The cables may be coupled to a quad-channel coupler, a preamplifier and an integrated, multiple conductor cable. Alternatively, all wires could be packaged or merged into a single unit or cord or plug (not illustrated) for insertion into the monitor for ease of management for the clinician and to prevent misplacement of wire plugs into wrong sockets.

The display and processing unit can display values at any one time for all non-invasive sensors being used. While the display and processing unit only displays amino acid levels for the embodiment described, the display and processing unit could easily display all values from, for example, three non-invasive sensors that are being used to monitor the parts of, say, the lower leg.

Additionally, interchangeability between different sensors for different functions (such as, for example, a leg sensor or an arm sensor or a cerebral sensor and a healthy sample sensor) could be recognized by the system and a set series of alarms or readings could be recorded based on the sensor inserted into the monitor or coupled to the monitor in a wireless manner. Therefore, the same monitor could be able to identify the type of sensor coupled to the monitor. A single monitor could be compatible with multiple different sensors and display appropriate data and alarms based on the sensor collecting data about the areas of interest of the human body.

Initial data from a patient with a suspected disease can be measured via, for example, amino acid levels in a diseased sample. If a disease is suspected, the amino acid levels can be monitored and would be deficient from an elevated state to an equal and then lower level with comparison to a healthy sample. Therefore, when comparing the diseased sample and the healthy sample, the diseased sample should show decreased amino acid levels. If levels begin to drop in the diseased sample compared to the healthy sample, an alarm or alert can be triggered to warn the medical practitioner. This alarm can be visual or audible (or both).

In a non-limiting preferred embodiment, a continuous analyte concentration (CAC) monitoring system can monitor amino acid levels. The CAC monitoring system can include a CPU of a display device that is coupled to non-invasive sensors. The CPU may also be coupled to a voice synthesizer and a speaker for providing status information and alarms to a medical practitioner.

The CPU can receive data from the CAC monitor in order to display amino acid levels. The CPU can activate an alarm, such as an audible or visual alarm (or both) with the voice synthesizer and speaker or displaying a warning message on the display device when the amino acid values of a patient drops. According to one non-limiting embodiment, in addition to activating an alarm, the CPU of the CAC monitoring system can increase a frequency of data collection for amino acid levels when a deficient amino acid level condition is detected by the continuous analyte concentration sensing system. In this way, the amino acid levels can be directly tracked and monitored by a medical practitioner. So, if the amino acid levels of a non-invasive sensor being tracked also start to drop, the CPU can sound an audible alarm and display a warning message to the medical practitioner to alert him or her of this changing condition.

Additionally, a running average of amino acid values over a certain time period can be calculated and displayed. The time period could be altered between multiple time periods from seconds to minutes to even hours. The purpose of the running average would be to limit the amount of variability of the amino acid values displayed on the screen. The current instantaneous value that is displayed in existing models is very labile. By using a running average, the trends can be monitored and the instantaneous changes can be smoothed out. This ability to decrease volatility would be important to prevent continual alert triggering if an alarm value was set by the medical practitioner.

In several embodiments, the continuous analyte concentration sensing system may be designed to allow for comparable values across a variety of patients by utilizing adjusted values. For example, short monitoring of a healthy sample may sometimes provide raw values from the continuous analyte concentration sensing system that may be very erratic and vary widely. However, continual monitoring with the continuous analyte concentration system may provide a baseline and allow a medical practitioner to adjust for values after the baseline is established. The continuous analyte concentration system 100 may have off-set values that may adjust readings for variations of age, demographics, health risks, high impact candidacy for diseases like PKU, MSUD, Tyrosinemia, and homocystinuria, as these may have relatively high prevalence among rare disease cases and significant morbidity can be mitigated with more frequent and precise monitoring.

### Non-Limiting Example Method for Monitoring Amino Acid Levels of a Human Body

A non-limiting example of a method for monitoring amino acid levels of a human body is described herein. The processes and operations of the inventive CAC monitoring system described below with respect to a logic flow that may include the manipulation of signals by a processor and the maintenance of these signals within data structures resident in one or more memory storage devices. For the purposes of this discussion, a process can be generally conceived to be a sequence of computer-executed steps leading to a desired result.

These steps usually require physical manipulations of physical quantities. Usually, though not necessarily, these quantities take the form of electrical, magnetic, or optical signals capable of being stored, transferred, combined, compared, or otherwise manipulated. It is convention for those skilled in the art to refer to representations of these signals as bits, bytes, words, information, elements, symbols, characters, numbers, points, data, entries, objects, images, files, or the like. It should be kept in mind, however, that these and similar terms are associated with appropriate physical quantities for computer operations, and that these terms are merely conventional labels applied to physical quantities that exist within and during operation of the computer.

It should also be understood that manipulations within the computer are often referred to in terms such as listing, creating, adding, calculating, comparing, moving, receiving, determining, configuring, identifying, populating, loading, performing, executing, storing etc. that are often associated with manual operations performed by a human operator. The operations described herein can be machine operations performed in conjunction with various input provided by a human operator or user that interacts with the computer. In addition, it should be understood that the programs, processes, methods, etc. described herein are not related or limited to any particular computer or apparatus. Rather, various types of general purpose machines may be used with the following process in accordance with the teachings described herein.

Embodiments of the present invention may comprise a computer program or hardware or a combination thereof which embodies the functions described herein.

However, it should be apparent that there could be many different ways of implementing the embodiments disclosed herein in computer programming or hardware design, and the invention should not be construed as limited to any one set of computer program instructions.

Further, a skilled programmer would be able to write such a computer program or identify the appropriate hardware circuits to implement the disclosed embodiments without difficulty based on the flow charts and associated description in the application text, for example. Therefore, disclosure of a particular set of program code instructions or detailed hardware devices is not considered necessary for an adequate understanding of how to make and use embodiments of the invention provided for herein.

Further, certain steps in the processes or process flow described in the logic flow must naturally precede others for the disclosed embodiments to function as described. However, the embodiments of the present invention disclosed herein are not limited to the order of the steps described if such order or sequence does not alter the functionality of the present invention. That is, it is recognized that some steps may be performed before, after, or in parallel other steps without departing from the scope and spirit of the present invention.

In several embodiments, the first step in the process for monitoring amino acid levels of a human body is described herein. In one step, a non-invasive sensor may be manufactured from sterile materials. Alternatively, a non-invasive sensor can be encapsulated with sterile materials so that it can be used in a, for example, surgical environment or so that it can be placed adjacent to or within particular areas of interest of the human body.

In one embodiment, a minimum amino acid value may be determined from a population of patients that indicates a healthy sample compared to a sample that may need to be investigated for disease. One of ordinary skill in the art recognizes that the embodiments of the invention provided for herein are not limited to patients having amino acid deficiencies. These embodiments may address any population of patients having a common medical trait and/or condition such as a disease so that a baseline level of amino acid values may be established for the patients having a common medical trait and/or condition and who need diagnosis of a disease or condition.

The CAC sensing system may further comprise a medicine delivery system that may operate in a manner similar to a drip line setup found in an ICU that would automatically administer medication to affect amino acid deficiency status. In one embodiment, the medicine delivery system may administer drugs. The system, via the medicine delivery system, may administer medicines automatically based on monitored conditions. For example, if the system via the continuous analyte concentration sensing system detects decreasing amino acid levels, then the central controller could issue a command to the medical delivery system to administer a drug. The medical delivery system may comprise a small pump for moving liquid medicines into a patient. This system could deliver medicines or other entities such as but not limited to intravenous fluid boluses or blood products.

In one embodiment, the CAC sensing system may be operably connected to the CAC electrode and configured to communicate with miniaturized electronics for on-device data processing, off-device data processing, edge artificial intelligence (Edge AI) capabilities. In some embodiments, the CAC sensing system may communicate with miniaturized electronics for wireless communication over Bluetooth to a smart phone or other base station to transfer data to a secure portal or secure cloud. In some embodiments, the data may be encrypted at rest, in transit or in use.

In a preferred embodiment, the medical practitioner may identify which sensors are monitoring a healthy sample and which sensors are monitoring a disease sample. The continuous analyte concentration sensing system may be designed to accommodate or to account for the different physiological states of disease. In one embodiment, the CAC sensing system may adjust its one or more monitoring algorithms depending upon the state of the human body. Also, in this step, the CAC sensing system may automatically identify which sample is healthy and which is diseased. The CAC sensing system may make these determinations based on detected sample characteristics. The CAC sensing system may then use a healthy sample as a control relative to the monitored disease sample as discussed above and below. The process then continues, and it may continue with additional steps.

In one embodiment, the CAC sensing system may contain a subcutaneous sensor and an Electronics cell. A subcutaneous sensor set can be provided for subcutaneous placement of an active portion of a flexible sensor, or the like, at a selected site in the body of a user. The subcutaneous or percutaneous portion of the sensor set may include a hollow, slotted insertion needle, and a cannula. The needle may be used to facilitate quick and easy subcutaneous placement of the cannula at the subcutaneous insertion site. Inside the cannula may be a sensing portion of the sensor to expose one or more sensor electrodes to the user's bodily fluids through a window formed in the cannula. In an embodiment of the invention, the one or more sensor electrodes may include a counter electrode, a reference electrode, and one or more electrodes. After insertion, the insertion needle is withdrawn to leave the cannula with the sensing portion and the sensor electrodes in place at the selected insertion site.

In particular embodiments, the subcutaneous sensor set facilitates accurate placement of a flexible thin film electrochemical sensor of the type used for monitoring specific blood parameters representative of a user's condition. The sensor may monitor glucose levels in the body and may be used in conjunction with automated or semi-automated medication infusion pumps of the external or implantable type as described, e.g., in U.S. Pat. Nos. 4,562,751; 4,678,408; 4,685,903 or 4,573,994, to control delivery of insulin to a diabetic patient. In one embodiment, the semi-automated medication infusion pumps of the external or implantable type can control delivery of other drugs given the diagnosis or health status of the patient.

Particular embodiments of the flexible electrochemical sensor are constructed in accordance with thin film mask techniques to include elongated thin film conductors embedded or encased between layers of a selected insulative material such as polyimide film or sheet, and membranes. The sensor electrodes at a tip end of the sensing portion may be exposed through one of the insulative layers for direct contact with patient blood or other body fluids, when the sensing portion (or active portion) of the sensor is subcutaneously placed at an insertion site. The sensing portion is joined to a connection portion that terminates in conductive contact pads, or the like, which are also exposed through one of the insulative layers. In alternative embodiments, other types of implantable sensors, such as chemical based, optical based, or the like, may be used.

In several embodiments, the connection portion and the contact pads are generally adapted for a direct wired electrical connection to a suitable monitor or sensor electronics device for monitoring a user's condition in response to signals derived from the sensor electrodes. Further description of flexible thin film sensors of this general type are be found in U.S. Pat. No. 5,391,250, entitled METHOD OF FABRICATING THIN FILM SENSORS, which is herein incorporated by reference. The connection portion may be conveniently connected electrically to the CAC monitor or Electronics cell or by a connector block (or the like) as shown and described in U.S. Pat. No. 5,482,473, entitled FLEX CIRCUIT CONNECTOR, which is also herein incorporated by reference. Thus, in accordance with embodiments of the present invention, subcutaneous sensor sets may be configured or formed to work with either a wired or a wireless characteristic monitor system.

In some embodiments, the sensor electrodes may be used in a variety of sensing applications and may be configured in a variety of ways. For example, the non-invasive sensor electrodes may be used in physiological parameter sensing applications in which some type of biomolecule is used as a catalytic agent. For example, the non-invasive sensor electrodes may be used in a glucose and oxygen sensor having a glucose oxidase (GOx) enzyme catalyzing a reaction with the non-invasive sensor electrodes. In one embodiment, the subcutaneous sensor electrodes, along with a biomolecule or some other catalytic agent, may be placed in a human body in a vascular or nonvascular environment. For example, the subcutaneous sensor electrodes and biomolecule may be placed in a vein and be subjected to a blood stream, or may be placed in a subcutaneous or peritoneal region of the human body.

In one embodiment, the CAC monitor 100 may include a power source, a sensor interface, processing electronics, and data formatting electronics. The CAC monitor 100 may be coupled to the sensor set by a cable through a connector that is electrically coupled to the connector block of the connection portion. In an alternative embodiment, the cable may be omitted. In this embodiment of the invention, the CAC monitor 100 may include an appropriate connector for direct connection to the connection portion of the sensor set. The sensor set may be modified to have the connector portion positioned at a different location, e.g., on top of the sensor set to facilitate placement of the CAC monitor 100 over the sensor set.

In embodiments of the invention, the sensor interface, the processing electronics, and the data formatting electronics are formed as separate semiconductor chips, however, alternative embodiments may combine the various semiconductor chips into a single or multiple customized semiconductor chips. The sensor interface connects with the cable that is connected with the sensor set.

The power source may be a battery. The battery can include three series silver oxide battery cells. In alternative embodiments, different battery chemistries may be utilized, such as lithium based chemistries, alkaline batteries, nickel metalhydride, or the like, and a different number of batteries may be used. The monitor may provide power to the sensor set via the power source, through the cable and cable connector. In an embodiment of the invention, the power is a voltage provided to the sensor set. In an embodiment of the invention, the power is a current provided to the sensor set. In an embodiment of the invention, the power is a voltage provided at a specific voltage to the sensor set.

In an embodiment of the invention, the first voltage and the second voltage may be positive voltages, or alternatively in other embodiments of the invention, negative voltages. In an embodiment of the invention, the first voltage may be positive and the second voltage may be negative, or alternatively, the first voltage may be negative and the second voltage may be positive. The first voltage may be different voltage levels for each of the iterations. In an embodiment of the invention, the first voltage may be a D.C. constant voltage. In other embodiments of the invention, the first voltage may be a ramp voltage, a sinusoid-shaped voltage, a stepped voltage, or other commonly utilized voltage waveforms. In an embodiment of the invention, the second voltage may be a D.C. constant voltage, a ramp voltage, a sinusoid-shaped voltage, a stepped voltage, or other commonly utilized voltage waveforms. In an embodiment of the invention, the first voltage or the second voltage may be an AC signal riding on a DC waveform. In an embodiment of the invention, the first voltage may be one type of voltage, e.g., a ramp voltage, and the second voltage may be a second type of voltage, e.g., a sinusoid-shaped voltage. In an embodiment of the invention, the first voltage (or the second voltage) may have different waveform shapes for each of the iterations. For example, if there are three cycles in a stabilization method, in a first cycle, the first voltage may be a ramp voltage, in the second cycle, the first voltage may be a constant voltage, and in the third cycle, the first voltage may be a sinusoidal voltage.

For example, analysis of the glucose (or analyte)-independent impedance data provides information on the efficiency of the sensor with respect to how quickly it hydrates and is ready for data acquisition using, e.g., values for 1 kHz real-impedance, 1 kHz imaginary impedance, and Nyquist Slope (to be described in more detail hereinbelow). Moreover, glucose-independent impedance data provides information on potential occlusion(s) that may exist on the sensor membrane surface, which occlusion(s) may temporarily block passage of glucose into the sensor and thus cause the signal to dip (using, e.g., values for 1 kHz real impedance). In addition, glucose-independent impedance data provides information on loss of sensor sensitivity during extended wear—potentially due to local oxygen deficit at the insertion site—using, e.g., values for phase angle and/or imaginary impedance at 1 kHz and higher frequencies. However, this is not meant to be limiting or required, as in some embodiments, phenylalanine concentrations are assessed. In still additional embodiments, any of the other 19 amino acids are assessed.

Within the context of electrode redundancy and EIS, a fusion algorithm may be used to take the diagnostic information provided by EIS for each redundant electrode and assess the reliability of each electrode independently. Weights, which are a measure of reliability, may then be added for each independent signal, and a single fused signal may be calculated that can be used to generate sensor glucose values as seen by the patient/subject.

As can be seen from the above, the combined use of redundancy, sensor diagnostics using EIS, and EIS-based fusion algorithms allows for an overall CAC sensing system that is more reliable than what is currently available. Redundancy is advantageous in at least two respects. First, redundancy removes the risk of a single point of failure by providing multiple signals. Second, providing multiple CAC electrodes where a single CAC electrode may be sufficient allows the output of the redundant CAC electrode to be used as a check against the primary CAC electrode, thereby reducing, and perhaps eliminating, the need for frequent calibrations. In addition, EIS diagnostics scrutinize the health of each CAC electrode autonomously without the need for a reference, in one example a glucose value (finger stick), thereby reducing the number of reference values required. However, the use of EIS technology and EIS diagnostic methods is not limited to redundant systems, i.e., those having more than one CAC electrode. Rather, as will be discussed below in connection with embodiments of the present invention, EIS may be advantageously used in connection with single- and/or multiple CAC electrode sensors.

EIS, or AC impedance methods, study the system response to the application of a periodic small amplitude AC signal. Here, where E is the applied potential, I is the current, and impedance (Z) is defined as ΔE/ΔI. However, although impedance, per se, may be mathematically simply defined as ΔE/ΔI, heretofore, there has been no commercialization success in application of EIS technology to continuous glucose monitoring. This has been due, in part, to the fact that glucose sensors are very complicated systems and, so far, no mathematical models have been developed which can completely explain the complexity of the EIS output for a glucose sensor.

One simplified electrical circuit model that has been used to describe electrochemical impedance spectroscopy. In one embodiment, IHP stands for Inner Helmholtz Plane, OHP stands for Outer Helmholtz Plane, CE is the counter electrode, WE is the working electrode, Cd is double layer capacitance, Rp is polarization resistance, Zw is Warburg impedance, and Rs is solution resistance. Each of the latter four components—double layer capacitance (Cd), Warburg impedance (Zw), polarization resistance (Rp), and solution resistance (Rs)—may play a significant role in sensor performance and can be measured separately by applying low- or high-frequency alternating working potential. For example, Warburg impedance is closely related to diffusional impedance of electrochemical systems, which is primarily a low-frequency impedance, and, as such, exists in all diffusion-limited electrochemical sensors. Thus, by correlating one or more of these components with one or more components and/or layers of a glucose sensor, one may use EIS technology as a sensor-diagnostics tool.

As is known, impedance may be defined in terms of its magnitude and phase, where the magnitude (|Z|) is the ratio of the voltage difference amplitude to the current amplitude, and the phase (θ) is the phase shift by which the current is ahead of the voltage. When a circuit is driven solely with direct current (DC), the impedance is the same as the resistant, i.e., resistance is a special case of impedance with zero phase angle. However, as a complex quantity, impedance may also be represented by its real and imaginary parts. In this regard, the real and imaginary impedance can be derived from the impedance magnitude and phase using the following equations:

$$Real Impedance ( ω ) = Magnitude ( ω ) \times cos ( Phase ( ω ) / 1 \; 80 \times π )$$

$$Imaginary Impedance ( ω ) = Magnitude ( ω ) \times sin ( Phase ( ω ) / 1 \; 80 \times π )$$

where ω represents the input frequency at which the magnitude (in ohms) and the phase (in degrees) are measured. The relationship between impedance, on the one hand, and current and voltage on the other (including how the former may be calculated based on measurement of the latter) will be explored more fully below in connection with the sensor electronics, including the Application Specific Integrated Circuit (ASIC), that has been developed for use in embodiments of the invention.

In one embodiment, the non-invasive sensor can be configured to periodically perform electrochemical measurements. In one preferred embodiment, the electrochemical measurements may be made by performing an Amperometric procedure. In one embodiment, the electrochemical measurements may be made by performing Potentiometry, Voltammetry, Cyclic Voltammetry, Differential Pulse Voltammetry, Amperometry, Chronoamperometry, Coulometry, Conductometry, or Galvanostatic Techniques.

In one embodiment, and out of various frequency ranges, the relatively-higher frequencies—i.e., those that fall generally between about 1 kHz and about 8 kHz—may be used to scrutinize the capacitive nature of the non-invasive sensor. Depending on the thickness and permeability of membranes, a typical range of impedance at the relatively-higher frequencies may be, e.g., between about 500 Ohms and 25 kOhms, and a typical range for the phase may be, e.g., between 0 degrees and −40 degrees. The relatively-lower frequencies (e.g., those that fall generally between about 0.1 Hz and about 100 Hz) on the other hand, are used to scrutinize the resistive nature of the non-invasive sensor. Here, depending on CAC electrode design and the extent of metallization, a typical functioning range for output real impedance may be, e.g., between about 50 kOhms and 300 kOhms, and a typical range for the phase may be between about −50 degrees to about −90 degrees.

Embodiments of the invention are also directed to an Analog Front End Integrated Circuit (AFE IC), which is a custom Application Specific Integrated Circuit (ASIC) that may provide the necessary analog electronics to: (i) support multiple potentiostats and interface with multi-terminal glucose sensors based on either Oxygen or Peroxide; (ii) interface with a microcontroller so as to form a micropower sensor system; and (iii) implement EIS diagnostics, fusion algorithms, and other EIS-based processes based on measurement of EIS-based parameters. More specifically, the ASIC incorporates diagnostic capability to measure the real and imaginary impedance parameters of the non-invasive sensor over a wide range of frequencies, as well as digital interface circuitry to enable bidirectional communication with the computing device. Moreover, the ASIC includes power control circuitry that enables operation at very low standby and operating power, and a real-time clock and a crystal oscillator so that an external computing device's power can be turned off.

Non-invasive sensors of preferred embodiments preferably exhibit 100±10% functionality, more preferably ~100% functionality over physiological glucose concentrations (from about 40 mg/dL to about 400 mg/dL) at oxygen concentrations preferably as low as about 0.6 mg/L or less, more preferably about 0.3 mg/L or less, more preferably still about 0.25 mg/L or less, even more preferably about 0.15 mg/L or less, even more preferably still about 0.1 mg/L or less, and most preferably about 0.05 mg/L or less. The non-invasive glucose sensors of preferred embodiments may typically consume 1 μg or less of enzyme over their operational lifetimes (typically 7 days or less).

In one embodiment, there may exist a molar excess of glucose relative to the amount of oxygen in blood; that is, for every free oxygen molecule in extracellular fluid, there are typically more than 100 glucose molecules present (see Updike et al., Diabetes Care 5:207-21(1982)). However, an immobilized enzyme-based non-invasive glucose sensors employing oxygen as co-reactant is preferably supplied with oxygen in non-rate-limiting excess in order for the sensor to respond linearly to changes in glucose concentration, while not responding to changes in oxygen concentration. Specifically, when a glucose-monitoring reaction is oxygen limited, glucose linearity may be lost at concentrations of glucose within a physiologically relevant range. Without a semipermeable membrane situated over the enzyme domain to control the flux of glucose and oxygen, a linear response to glucose levels can be obtained only for glucose concentrations that are less than physiologically relevant concentrations, i.e., less than 40 mg/dL. See, e.g., Luong, J H et al., Characterization of interacting ferrocene-cyclodextrin systems and their role in mediated biosensors, J. Mol. Recognit. 1995 Jan. 8 (1-2), 132-138. However, in a clinical setting, a linear response to glucose levels may be desirable up to at least about 400 mg/dL. The sensors of preferred embodiments may, typically, exhibit substantial linearity (e.g., r2 of 0.95 or greater in vitro) at physiologically relevant concentrations from about 40 mg/dL or less up to about 400 mg/dL glucose or more.

Although the present disclosure references glucose monitoring by way of example, it should be understood that the systems, methods, devices, and techniques described herein are not limited to glucose monitoring alone. Unless otherwise expressly stated or the context clearly dictates otherwise, any reference to glucose monitoring, glucose detection, glucose concentration measurement, or any related glucose-sensing functionality shall be understood to apply equally, mutatis mutandis, to the monitoring, detection, and concentration measurement of phenylalanine and any of the other nineteen naturally occurring amino acids, including alanine, arginine, asparagine, aspartic acid, cysteine, glutamic acid, glutamine, glycine, histidine, isoleucine, leucine, lysine, methionine, proline, serine, threonine, tryptophan, tyrosine, and valine. Accordingly, where a particular embodiment is described in the context of glucose monitoring, one of ordinary skill in the art will appreciate that such embodiment may be adapted, without undue experimentation, to monitor, detect, or measure the concentration of phenylalanine or any other amino acid, either individually or in combination. The use of glucose as a non-limiting analyte throughout this disclosure is intended solely for clarity and ease of illustration and shall not be construed as limiting the scope of the claims or the breadth of the inventive concepts disclosed herein.

It is noted that the techniques described herein may be embodied in executable instructions stored in a computer readable medium for use by or in connection with a processor-based instruction execution machine, system, apparatus, or device. It will be appreciated by those skilled in the art that, for some embodiments, various types of computer-readable media can be included for storing data. As used herein, a “computer-readable medium” includes one or more of any suitable media for storing the executable instructions of a computer program such that the instruction execution machine, system, apparatus, or device may read (or fetch) the instructions from the computer-readable medium and execute the instructions for carrying out the described embodiments. Suitable storage formats include one or more of an electronic, magnetic, optical, and electromagnetic format. A non-exhaustive list of conventional exemplary computer-readable medium includes: a portable computer diskette; a random-access memory (RAM); a read-only memory (ROM); an erasable programmable read only memory (EPROM); a flash memory device; and optical storage devices, including a portable compact disc (CD), a portable digital video disc (DVD), and the like.

It should be understood that the arrangement of components illustrated in the attached Figures are for illustrative purposes and that other arrangements are possible. For example, one or more of the elements described herein may be realized, in whole or in part, as an electronic hardware component. Other elements may be implemented in software, hardware, or a combination of software and hardware. Moreover, some or all of these other elements may be combined, some may be omitted altogether, and additional components may be added while still achieving the functionality described herein. Thus, the subject matter described herein may be embodied in many different variations, and all such variations are contemplated to be within the scope of the claims.

To facilitate an understanding of the subject matter described herein, many aspects are described in terms of sequences of actions. It will be recognized by those skilled in the art that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar references in the context of describing the subject matter (particularly in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. Furthermore, the foregoing description is for the purpose of illustration only, and not for the purpose of limitation, as the scope of protection sought is defined by the claims as set forth hereinafter together with any equivalents thereof. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

With respect to ingredients that are grouped by function or characterized herein, unless otherwise expressly stated herein, it is not precluded that such an ingredient could be grouped with or characterized by another function. For example, an ingredient discussed herein as a solubilizer could also function as a surfactant and vice versa. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” Language such as “at least 95%” (S)-nicotine in the form of a free base alkaloid includes 96%, 97%, 98%, 99%, and 100% (S)-nicotine in the form of a free base alkaloid. Any titles or subheadings used herein are for organization purposes and should not be used to limit the scope of embodiments disclosed herein.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

What is claimed is:

1. A system for monitoring analyte concentration levels, the system comprising:
an integrated circuit chip configured to classify data representing a level of an amino acid as representing an amino acid deficient condition or an amino acid non-deficient condition, the integrated circuit chip comprising:
an analog front end cell including an amplifier cell in communication with a continuous analyte concentration electrode of a non-invasive sensor and an analog signal processing unit cell in communication with the amplifier cell;
a digital back end channel controller cell;
a feature extraction engine processor cell in communication with the digital back end channel controller cell and the analog front end cell; and
a classification engine cell in communication with the feature extraction engine processor cell and the digital back end channel controller cell, wherein the classification engine cell comprises a patient-specific support vector machine configured to process a signal received from the continuous analyte concentration electrode and designate the signal as representing an amino acid deficient condition or an amino acid non-deficient condition; and
an output component configured to output a recommendation to administer amino acid if the integrated circuit chip classifies the measurement data as representing the amino acid deficient condition, or a recommendation not to administer amino acid if the integrated circuit chip classifies the measurement data as representing the amino acid non-deficient condition.

2. The system of claim 1, wherein the amplifier cell comprises a chopper stabilized-capacitive coupled instrumentation amplifier cell.

3. The system of claim 1, wherein the amplifier cell comprises an autozeroing amplifier circuit cell.

4. The system of claim 1, further comprising an analog to digital converter cell in communication with the analog signal processing unit cell and the feature extraction engine processor cell.

5. The system of claim 4, wherein the analog to digital converter cell comprises a successive approximation register analog-to-digital converter cell.

6. The system of claim 1, wherein the feature extraction engine processor cell comprises a plurality of bandpass filter cells configured to divide a signal from the analog front end cell into a plurality of sub-bands and a plurality of integrator cells configured to integrate respective ones of the plurality of sub-bands to determine respective sub-band energies and aggregate the respective sub-band energies into a feature vector.

7. The system of claim 6, wherein the feature extraction engine processor cell further comprises a decimation filter cell in communication between the analog front end cell and the plurality of bandpass filter cells.

8. The system of claim 1, wherein the analog front end cell is one of a plurality of analog front end cells, wherein the feature extraction engine processor cell is one of a plurality of feature extraction engine processor cells, and wherein the integrated circuit chip further comprises a multiplexer cell in communication with the plurality of analog front end cells and an analog to digital converter cell in communication with the multiplexer cell and the plurality of feature extraction engine processor cells.

9. The system of claim 1, wherein the feature extraction engine processor cell comprises a plurality of bandpass filter cells and a plurality of integrator cells configured to determine respective sub-band energies.

10. The system of claim 1, wherein the integrated circuit chip further comprises a static random-access memory, an external memory interface, and an external data interface.

11. A method for monitoring a continuous analyte concentration using an integrated circuit chip, the method comprising:
amplifying a signal from a continuous analyte concentration electrode of a non-invasive sensor to generate an amplified analyte concentration signal;
adapting gain of the amplified analyte concentration signal using an analog signal processing unit to generate an adapted gain signal;
digitizing the adapted gain signal using an analog to digital converter;
extracting a feature vector from the digitized adapted gain signal;
designating, with a patient-specific support vector machine classification engine of the integrated circuit chip, the feature vector as representing an amino acid deficient condition or an amino acid non-deficient condition; and
outputting a recommendation to administer amino acid if the integrated circuit chip classifies measurement data as representing the amino acid deficient condition, or a recommendation not to administer amino acid if the integrated circuit chip classifies the measurement data as representing the amino acid non-deficient condition.

12. The method of claim 11, wherein extracting the feature vector comprises dividing the digitized adapted gain signal into a plurality of sub-bands, integrating respective ones of the plurality of sub-bands to calculate a respective energy for respective ones of the plurality of sub-bands, and aggregating the energies into the feature vector.

13. The method of claim 12, wherein extracting the feature vector further comprises decimating the digitized adapted gain signal before dividing the digitized adapted gain signal into the plurality of sub-bands.

14. The method of claim 11, wherein designating the feature vector comprises generating, in response to patient data, a hyperplane separating a deficient region of feature hyperspace from a non-deficient region of feature hyperspace and determining in which of the deficient region or the non-deficient region the feature vector exists.

15. The method of claim 11, further comprising comparing amino acid levels from a diseased sample with amino acid levels from a healthy sample and triggering an alarm when the amino acid levels from the diseased sample decrease relative to the amino acid levels from the healthy sample.

16. The method of claim 11, further comprising increasing a frequency of data collection when the amino acid deficient condition is detected.

17. A device comprising:
an integrated circuit chip for continuous analyte concentration monitoring, the integrated circuit chip comprising:
an analog front end cell including an amplifier cell in communication with a continuous analyte concentration electrode of a non-invasive sensor and an analog signal processing unit cell in communication with the amplifier cell;
a digital back end channel controller cell;
a feature extraction engine processor cell in communication with the digital back end channel controller cell and the analog front end cell; and
a classification engine cell in communication with the feature extraction engine processor cell and the digital back end channel controller cell, wherein the classification engine cell comprises a patient-specific support vector machine configured to process a signal received from the continuous analyte concentration electrode and designate the signal as representing an amino acid deficient condition or an amino acid non-deficient condition; and
an output component configured to output a recommendation to administer amino acid if the integrated circuit chip classifies the measurement data as representing the amino acid deficient condition, or a recommendation not to administer amino acid if the integrated circuit chip classifies the measurement data as representing the amino acid non-deficient condition.

18. The device of claim 17, wherein the amplifier cell comprises a chopper stabilized-capacitive coupled instrumentation amplifier cell.

19. The device of claim 17, wherein the amplifier cell comprises an autozeroing amplifier circuit cell.

20. The device of claim 17, further comprising an analog to digital converter cell in communication with the analog signal processing unit cell and the feature extraction engine processor cell.
